(19) United States
(12) Patent Application Publication
(10) Pub. No.: US 2026/0068921 A1
(43) Pub. Date: Mar. 12, 2026

(54) ORAL POUCH PRODUCT

(71) Applicant: Altria Client Services LLC, Richmond, VA (US)

(72) Inventors: Jeanie BAILEY, Richmond, VA (US); Baiwei CHEN, Glen Allen, VA (US); Matthew LAMPE, New Kent, VA (US); Tanmay DESAI, Henrico, VA (US); Dwight WILLIAMS, Powhatan, VA (US)

(73) Assignee: Altria Client Services LLC, Richmond, VA (US)

(21) Appl. No.: 19/324,600

(22) Filed: Sep. 10, 2025

Related U.S. Application Data
(60) Provisional application No. 63/693,903, filed on Sep. 12, 2024.

(51) Int. Cl. A24B 13/00; A24B 15/16; A61K 9/00; A61K 31/522; B65B 9/20; B65D 75/46

(52) U.S. Cl. CPC A24B 13/00; A24B 15/16; A61K 9/009; A61K 31/522; B65B 9/20; B65D 75/46

(57) ABSTRACT

An oral pouch product for oral use may include a filler and a pouch enclosing the filler. The pouch may include a first edge and a second edge extending in parallel, a first sealed edge extending between the first edge and the second edge, and a second sealed edge extending between the first edge and the second edge. The second sealed edge may be spaced apart from the first sealed edge. The first sealed edge may be oriented such that the first sealed edge forms an acute angle with the first edge and an obtuse angle with the second edge.

### CROSS-REFERENCE TO RELATED APPLICATIONS

This application claims the benefit of U.S. Provisional Application No. 63/693,903, filed on Sep. 12, 2024, the entire disclosure of which is incorporated herein by reference in its entirety.

### BACKGROUND

This disclosure generally relates to oral pouch products and methods of manufacturing an oral pouch product.

### SUMMARY

In at least one example embodiment, a pouch product for oral use may include a filler and a pouch enclosing the filler. The pouch may include a first edge and a second edge extending in parallel, a first sealed edge extending between the first edge and the second edge, and a second sealed edge extending between the first edge and the second edge. The second sealed edge may be spaced apart from the first sealed edge. The first sealed edge may be oriented such that the first sealed edge forms an acute angle with the first edge and an obtuse angle with the second edge.

In at least one example embodiment, the acute angle is 45 degrees or less.

In at least one example embodiment, the acute angle ranges from 20 to 45 degrees.

In at least one example embodiment, the first sealed edge is parallel to the second sealed edge.

In at least one example embodiment, the pouch includes paper.

In at least one example embodiment, the filler has a weight of 250 mg to 700 mg.

In at least one example embodiment, a distance between the first edge and the second edge is from 8 mm to 12 mm.

In at least one example embodiment, the distance between the first edge and the second edge is less than a length of the first edge.

In at least one example embodiment, the pouch has a longest dimension of 29 mm to 36 mm.

In at least one example embodiment, the filler includes tobacco.

In at least one example embodiment, the filler includes nicotine.

In at least one example embodiment, the filler includes caffeine.

In at least one example embodiment, the first sealed edge forms an oblique angle with respect to the second edge.

In at least one example embodiment, the second sealed edge is perpendicular to the first edge and the second edge.

In at least one example embodiment, an oral pouch product includes a tobacco filler and pouch enclosing the tobacco filler. The pouch may include a first edge and a second edge extending in parallel, a first sealed edge extending between the first edge and the second edge, and a second sealed edge extending between the first edge and the second edge. The second sealed edge may be spaced apart from the first sealed edge. The first sealed edge may be oriented such that the first sealed edge forms an acute angle with the first edge and an obtuse angle with the second edge. The first sealed edge may extend in parallel to the second sealed edge, and the acute angle may range from 20 to 45 degrees.

In some example embodiments, the second sealed edge is oriented such that the second sealed edge forms an acute angle with the second edge and an obtuse angle with the first edge.

In at least one example embodiment, an oral pouch product for oral use includes a filler; and a pouch enclosing the filler, the pouch including, a first edge and a second edge extending in parallel, a first sealed edge extending between the first edge and the second edge, and a second sealed edge extending between the first edge and the second edge, the second sealed edge spaced apart from the first sealed edge, the first sealed edge oriented such that the first sealed edge forms a non-perpendicular angle with at least one of the first edge or the second edge.

In at least one example embodiments, the first sealed edge forms a first non-perpendicular angle with the first edge and a second non-perpendicular angle with the second edge.

In at least one example embodiment, wherein the second sealed edge is oriented such that the second sealed edge forms a non-perpendicular angle with at least one of the first edge and the second edge.

In at least one example embodiment, wherein the second sealed edge forms a third non-perpendicular angle with the first edge and a fourth non-perpendicular angle with the second edge.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1A is a perspective view of an oral pouch product according to at least one example embodiment.

FIG. 1B is a perspective view of an oral pouch product according to at least one example embodiment.

FIG. 1C is a perspective view of an oral pouch product according to at least one example embodiment.

FIG. 2A is a perspective view of an oral pouch product according to at least one example embodiment.

FIG. 2B is a perspective view of an oral pouch product according to at least one example embodiment.

FIG. 2C is a perspective view of an oral pouch product according to at least one example embodiment.

FIG. 3 is a cross-sectional view along line A-A of FIG. 1A of an oral pouch product according to at least one example embodiment.

FIG. 4 depicts a method of manufacturing an oral pouch product according to at least one example embodiment.

FIG. 5 is a flowchart illustrating a method of manufacturing an oral pouch product according to at least one example embodiment.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc., may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” “inside,” “outside,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

Example embodiments are described herein with reference to cross-sectional illustrations that are schematic illustrations of example embodiments. As such, variations from the shapes of the illustrations are to be expected. Thus, example embodiments should not be construed as limited to the shapes of regions illustrated herein but are to include deviations and variations in shapes. When the terms “about” or “substantially” are used in connection with a numerical value, it is intended that the associated numerical value include a tolerance of ±10% around the stated numerical value unless the context indicates otherwise.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein. Elements identified by the same reference numeral may be substantially the same unless specified or described otherwise.

In at least one example embodiment, the oral pouch product is an oral tobacco product, an oral non-tobacco product, an oral cannabis product, or any combination thereof. The oral pouch product may include loose material (e.g., loose cellulosic material), shaped material (e.g., plugs or twists), pouched material, tablets, lozenges, chews, gums, films, any other oral product, or any combination thereof.

In at least one example embodiment, a pouch for enhancing the product experience for an adult consumer may have slanted ends in order to more comfortably fit into the mouth of an adult consumer.

The oral pouch product may include chewing tobacco, snus, moist snuff tobacco, dry snuff tobacco, other smokeless tobacco and non-tobacco products for oral consumption, or any combination thereof.

Where the oral pouch product is an oral tobacco product including smokeless tobacco product, the smokeless tobacco product may include tobacco that is whole, shredded, cut, granulated, reconstituted, cured, aged, fermented, pasteurized, or otherwise processed. Tobacco may be present as whole or portions of leaves, flowers, roots, stems, extracts (e.g., nicotine), or any combination thereof.

In at least one example embodiment, the oral pouch product includes a tobacco extract, such as a tobacco-derived nicotine extract, and/or synthetic nicotine. The oral pouch product may include nicotine alone or in combination with a carrier (e.g., white snus), such as a cellulosic material. The carrier may be a non-tobacco material (e.g., microcrystalline cellulose) or a tobacco material (e.g., tobacco fibers having reduced or eliminated nicotine content, which may be referred to as “exhausted tobacco plant tissue or fibers”). In some example embodiments, the exhausted tobacco plant tissue or fibers can be treated to remove at least 25%, 40%, 50%, 60%, 70%, 75%, 80%, 85%, 90%, or 95% of the nicotine. For example, the tobacco plant tissue can be washed with water or another solvent to remove the nicotine.

In other example embodiments, the oral pouch product may include cannabis, such as cannabis plant tissue and/or cannabis extracts. In at least one example embodiment, the cannabis material includes leaf and/or flower material from one or more species of cannabis plants and/or extracts from the one or more species of cannabis plants. The one or more species of cannabis plants may include Cannabis sativa, Cannabis indica, and/or Cannabis ruderalis. In at least one example embodiment, the cannabis may be in the form of fibers. In at least one example embodiment, the cannabis may include a cannabinoid, a terpene, and/or a flavonoid. In at least one example embodiment, the cannabis material may be a cannabis-derived cannabis material, such as a cannabis-derived cannabinoid, a cannabis-derived terpene, and/or a cannabis-derived flavonoid.

The oral pouch product (e.g., the oral tobacco product, the oral non-tobacco product, or the oral cannabis product) may have various ranges of moisture. In at least one example embodiment, the oral pouch product is a dry oral pouch product having a moisture content ranging from 5% by weight to 10% by weight. In at least one example embodiment, the oral pouch product has a medium moisture content, such as a moisture content ranging from 20% by weight to 35% by weight. In at least one example embodiment, the oral pouch product is a wet oral pouch product having a moisture content ranging from 40% by weight to 55% by weight.

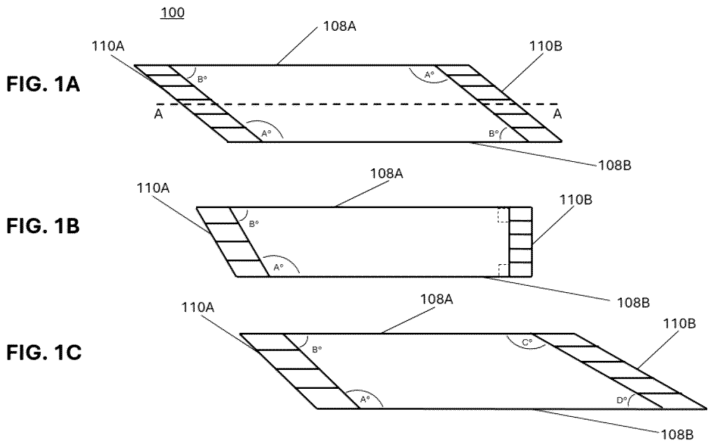

FIG. 1A is a perspective view of an oral pouch product according to at least one example embodiment.

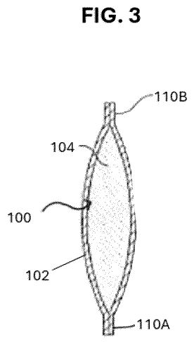

In at least one example embodiment, as shown in FIG. 1 an oral pouch product 100 is configured to fit in an adult consumer's mouth. The oral pouch product 100 may include a pouch wrapper 102 and a pouch filler 104 (as will be further discussed with respect to FIG. 3). The pouch filler 104 may be within a cavity defined by the pouch wrapper 102 in between a first sealed edge 110A and a second sealed edge 110B corresponding to opposite ends.

The oral pouch product 100 may have a first edge 108A and a second edge 108B opposite the first edge 108A. The first edge 108A and the second edge 108B may extend in parallel to each other in a first direction defining a longest dimension of the oral pouch product 100. The oral pouch product 100 may include a first sealed edge 110A and a second sealed edge 110B. The first sealed edge 110A and the second sealed edge 110B may extend in a second direction transverse to the first edge 108A and the second edge 108B. In at least one example embodiment, the first sealed edge 110A and the second sealed edge 110B may form or intersect the first edge 108A and the second edge 108B at oblique angles (e.g., non-perpendicular angles). For example, in some example embodiment, the first sealed edge 110A may form or intersect at least one of the first edge 108A or the second edge 108B at a non-perpendicular angle. In some example embodiments, the second sealed edge 110B may form or intersect at least one of the first edge 108A or the second edge 108B at a non-perpendicular angle. In some example embodiments, the first sealed edge 110A and the second sealed edge 110B may both intersect both the first edge 108A and the second edge 108B at non-perpendicular angles.

In at least one example embodiment, the first sealed edge 110A may form or intersect the second edge 108B at an obtuse angle. In at least one example embodiment, the first sealed edge 110A may form or intersect the first edge 108A at an acute angle. In at least one example embodiment, the second sealed edge 110B may form or intersect the first edge 108A at an acute angle. In at least one example embodiment, the second sealed edge 110B may form or intersect the second edge 108B at an obtuse angle. Collectively, the first edge 108A, the second edge 108B, the first sealed edge 110A, and the second sealed edge 110B may define the boundaries of the oral pouch product 100 in a first direction and a second direction. For example, the first sealed edge 110A and/or the second sealed edge 110B may extend at an incline from the first edge 108A to the second edge 108B. For example, a degree of incline of the first sealed edge 110A and/or the second sealed edge 110B may extend at a constant degree of incline with respect to the first edge 108A and/or the second edge 108B.

In at least one example embodiment, the first sealed edge 110A may form an acute angle B with the first edge 108A. For example, the acute angle B may be less than 50 degrees (e.g., less than 45 degrees, less than 40 degrees, less than 35 degrees, less than 30 degrees, e.g., less than 25 degrees). Additionally, the acute angle B may be greater than 15 degrees (e.g., greater than 20 degrees, greater than 25 degrees, greater than 30 degrees, greater 35 degrees, greater than 40 degrees). The acute angle B may be between 45 degrees and 20 degrees (e.g. between 40 degrees and 25 degrees, between 35 degrees and 30 degrees).

In at least one example embodiment, the second sealed edge 110B may form an obtuse angle A with the first edge 108A. For example, the obtuse angle A may be less than 165 degrees (e.g., less than 160 degrees, less than 155 degrees, less than 150 degrees, less than 145 degrees, e.g., less than 140 degrees). Additionally, the obtuse angle A may be greater than 130 degrees (e.g., greater than 135 degrees, greater than 140 degrees, greater than 145 degrees, greater 150 degrees, greater than 155 degrees). The obtuse angle A may be between 160 degrees and 135 degrees (e.g. between 155 degrees and 140 degrees, between 150 degrees and 145 degrees).

In at least one example embodiment, the first sealed edge 110A may form an obtuse angle A with the second edge 108B. For example, the obtuse angle A may be less than 165 degrees (e.g., less than 160 degrees, less than 155 degrees, less than 150 degrees, less than 145 degrees, e.g., less than 140 degrees). Additionally, the obtuse angle A may be greater than 130 degrees (e.g., greater than 135 degrees, greater than 140 degrees, greater than 145 degrees, greater 150 degrees, greater than 155 degrees). The obtuse angle A may be between 160 degrees and 135 degrees (e.g. between 155 degrees and 140 degrees, between 150 degrees and 145 degrees).

In at least one example embodiment, the second sealed edge 110B may form an acute angle B with the second edge 108B. For example, the acute angle B may be less than 50 degrees (e.g., less than 45 degrees, less than 40 degrees, less than 35 degrees, less than 30 degrees, e.g., less than 25 degrees). Additionally, the acute angle B may be greater than 15 degrees (e.g., greater than 20 degrees, greater than 25 degrees, greater than 30 degrees, greater 35 degrees, greater than 40 degrees). The acute angle B may be between 45 degrees and 20 degrees (e.g. between 40 degrees and 25 degrees, between 35 degrees and 30 degrees).

In at least one example embodiment, the acute angle B formed between the first sealed edge 110A and the first edge 108A may be equal to the acute angle B formed between the second sealed edge 110B and the second edge 108B. In at least one example embodiment, the obtuse angle A formed between the first sealed edge 110A and the second edge 108B may be equal to the obtuse angle A formed between the second sealed edge 110B and the first edge 108A. That is to say, in at least one example embodiment, as described above with regard to FIG. 1A, the first sealed edge 110A and the second sealed edge 110B may extend in parallel to each other. However, the example embodiments are not so limited thereto. For example, the first sealed edge 110A and the second sealed edge 110B may not extend in parallel to each other as described in more detail below with regard to FIGS. 1B-1C.

By angling the first sealed edge 110A and the second sealed edge 110B of the oral pouch product 100, the oral pouch product 100 may more comfortably fit in the mouth of an adult consumer. For example, in the operation of an oral pouch product 100 an adult consumer may place the oral pouch product between the gums and the cheek of the adult consumer. Accordingly, an oral pouch product having an angled edge may more closely conform to the natural contours of the mouth of the adult consumer and thereby provide an adult consumer with a more comfortable consumer experience.

FIG. 1B is a perspective view of an oral pouch product according to at least one example embodiment.

In at least one example embodiment, for example as depicted in FIG. 1B, the first sealed edge 110A may extend at an angle oblique to the first edge 108A and the second edge 108B. However, the second sealed edge 110B may be perpendicular to the first edge 108A and the second edge 108B.

FIG. 1C is a perspective view of an oral pouch product according to at least one example embodiment.

In at least one example embodiment, for example as depicted in FIG. 1C, the first sealed edge 110A may extend in a first direction transverse to the first edge 108A and the second edge 108B such that the first sealed edge 110A forms a first complementary pair of oblique angles with respect to the first edge 108A and the second edge 108B. The second sealed edge 110B may extend in a second direction different from the first direction. The second direction may be transverse to the first edge 108A and the second edge 108B such that the second sealed edge 110B forms a second pair of complementary oblique angles with respect to the first edge 108A and the second edge 108B different from the first pair of complementary oblique angles. For example, the first sealed edge 110A may extend in a first direction that forms an oblique angle with respect to the second sealed edge 110B extending in a second direction.

In at least one example embodiment, the first sealed edge 110A may form an obtuse angle A with respect to the second edge 108B and an acute angel B with respect to the first edge 108A. The second sealed edge 110B may form an obtuse angle C with respect to the first edge 108A and an acute angle D with respect to the second edge 108B. Angle A, angle B, angle C, and angle D may all be different in degree. Acute angle B and acute angle D may be any degree described above with regard to acute angle B described with reference to FIG. 1A. Obtuse angle A and obtuse angle C may be any degree described above with regard obtuse angle A described with reference to FIG. 1A.

For example, by angling one of the sealed edges 110A or 110B to be more acute with respect to the first edge 108A or the second edge 108B the oral pouch product 100 may more comfortably fit within the natural contours at the back of the mouth of an adult consumer. Additionally, by having the opposing sealed edge form a different angle (e.g., closer to 90 degrees with respect to the first edge 108A or the second edge 108B) the pointedness of corners may be reduced, further increasing the comfort of an adult consumer.

The distance between the first edge 108A and the second edge 108B may be uniform. For example, the distance between the first edge 108A and the second edge 108B may be between about 8 mm to about 12 mm. For example, a width of the oral pouch product 100 may be about 8 mm. For example, a width of the oral pouch product 100 may be about 9 mm. For example, a width of the oral pouch product 100 may be about 10 mm. For example, a width of the oral pouch product 100 may be about 11 mm. For example, a width of the oral pouch product 100 may be about 12 mm.

The oral pouch product 100 may have a longest dimension, for example a length, between a tip of the first sealed edge 110A and the second sealed edge 110B between about 29 mm and about 36 mm. For example, the oral pouch product may have a longest dimension of about 29 mm. For example, the oral pouch product may have a longest dimension of about 30 mm. For example, the oral pouch product may have a longest dimension of about 31 mm. For example, the oral pouch product may have a longest dimension of about 32 mm. For example, the oral pouch product may have a longest dimension of about 33 mm. For example, the oral pouch product may have a longest dimension of about 34 mm. For example, the oral pouch product may have a longest dimension of about 35 mm. For example, the oral pouch product may have a longest dimension of about 36 mm.

In at least one example embodiment, the pouch wrapper 102 may be formed from a material that is generally recognized as safe (“GRAS”) for use and/or contact with food. The material may be stain resistant, water permeable, and/or porous. The pouch wrapper may have a thickness of about 0.05 mm to about 0.25 mm (e.g., about 0.075 mm to about 0.100 mm or about 0.1 mm to about 0.20 mm). The pouch wrapper 102 can be formed of a permeable material or a semi-permeable material, such that, for example, saliva, water, or both saliva and water can pass through the pouch wrapper 102 and into the inner cavity defined by the pouch wrapper 102. Flavors and juices formed by mixing saliva and/or water with the pouch filler 104 contained within the oral pouch product 100 can be drawn out of the oral pouch product 100 through the pouch wrapper 102.

In at least one example embodiment, the pouch wrapper 102 includes a non-woven material. The non-woven material may be formed of a polymer, including one or more synthetic and/or natural polymers. For example, pouch wrapper 102 may be formed of a mesh material formed of spun or melt-blown fibers, such as polyurethane fibers as described in U.S. Pat. Nos. 10,448,669, 10,463,070, and/or 9,414,624, the entire contents of each of which is incorporated herein by reference thereto. The mesh material may be at least partially elastomeric. The mesh material may be white and may exclude any colorant. In other example embodiments, the mesh material may include a colorant. Further, because of the material used to form the wrapper 102, the oral pouch product 100 may exclude seams so as to provide a softer pouch.

In at least one example embodiment, the mesh material has a basis weight of greater than or equal to about 10 grams per square meter (“gsm”) (e.g., greater than or equal to about 15 gsm, greater than or equal to about 20 gsm, greater than or equal to about 25 gsm, greater than or equal to about 30 gsm, greater than or equal to about 35 gsm, greater than or equal to about 40 gsm, or greater than or equal to about 45 gsm). The basis weight may be less than or equal to about 50 gsm (e.g., less than or equal to about 45 gsm, less than or equal to about 40 gsm, less than or equal to about 35 gsm, less than or equal to about 30 gsm, less than or equal to about 25 gsm, less than or equal to about 20 gsm, or less than or equal to about 15 gsm). In at least one example embodiment, the basis weight ranges from about 20 gsm to about 30 gsm.

In at least one other example embodiment, the pouch wrapper 102 comprises a paper. For example, the pouch wrapper 102 may be formed of a cellulose fiber material, such as tea bag material or other materials typically used to form snus pouches. In at least one example embodiment, the pouch wrapper 102 has a desired (or alternatively, predetermined) level for basis weight and/or wet strength to reduce occurrence of breakage of the pouch wrapper 102 during manufacturing operations, storage, and placement in an adult consumer's mouth. For example, the pouch wrapper 102 may comprise a tea bag material having a basis weight of about 16.5 gsm with a wet tensile CD strength of 68 N/m. In another example embodiment, the pouch wrapper 102 may be formed of a paper having a wet MD tensile strength of about 45 N/mm to about 52 N/mm.

In at least one example embodiment, the pouch wrapper 102 may include a hydrophobic paper or material. The hydrophobic paper may be formed of a cellulosic material. The hydrophobic paper may be non-woven material and may include any hydrophobic materials. The hydrophobic materials may be synthetic materials and/or semi-synthetic materials. The hydrophobic materials may include viscose, rayon, lyocell, and/or modal fibers. The pouch wrapper 102 may be treated to make the pouch wrapper 102 hydrophobic. In other example embodiments, the hydrophobic material may be a woven material.

In at least one example embodiment, the oral pouch product 100 may include between about 250 mg and about 700 mg of pouch filler 104 (e.g., between 300 mg and 650 mg, between 350 mg and 600 mg, between 400 mg and 550 mg, between 450 mg and 500 mg).

FIG. 3 is a cross-sectional view along line A-A of FIG. 1A of an oral pouch product according to at least one example embodiment.

As depicted in FIG. 3 a cavity defined by pouch wrapper 102 may include a pouch filler 104.

In at least one example embodiment, the pouch filler 104 may include one or more elements such as a mouth-stable polymer, a mouth-soluble polymer, a sweetener (e.g., a synthetic sweetener and/or a natural sweetener), an energizing agent, a soothing agent (e.g., theanine and/or melatonin), a focusing agent (e.g., gingko biloba), a plasticizer, mouth-soluble or partially-soluble fibers (e.g., sugar beet fibers), an alkaloid, a mineral, a vitamin, a dietary supplement, a nutraceutical, a coloring agent, an amino acid, a chemesthetic agent, an antioxidant, a food-grade emulsifier, a pH modifier, a botanical (e.g., green tea), a tooth-whitening agent (e.g., sodium hexametaphosphate (SHMP)), a therapeutic agent, a processing aid, a stearate (e.g., magnesium and/or potassium), a wax (e.g., glycerol monostearate, propylene glycol monostearate, and/or an acetylated monoglyceride), a stabilizer (e.g., ascorbic acid and monosterol citrate, butylated hydroxytoluene (BHT), or butylated hydroxyanisole (BHA)), a lubricant (e.g., sodium lauryl sulfate (SLS)), a disintegrating agent, a lubricant, a preservative (e.g., sodium benzoate), a filler, a flavorant, an effervescent (e.g., carbon dioxide embedded in a flavorant or a filler), flavor masking agents, a bitterness receptor site blocker, a receptor site enhancers, other additives, or any combination thereof. The pouch filler 104 may include multiple additional elements. Additionally, a single element may belong to more than one of the categories above.

As used herein, “mouth-soluble” means that the polymer experiences significant degradation when exposed to saliva within an oral cavity of an adult consumer over a period of about four hours. In at least one example embodiment, the mouth-soluble polymer disintegrates when exposed to saliva having a normal human body temperature (i.e., 98.6° F.) for a period of less than or equal to about an hour (e.g., less than or equal to about 30 minutes, less than or equal to about 15 minutes, less than or equal to about 10 minutes, or less than or equal to about 5 minutes).

In at least one example embodiment, the pouch filler 104 includes a mouth-soluble polymer. The mouth-soluble polymer may include, for example, a cellulosic polymer, a natural polymer, a seaweed-derived polymer, a microbial-derived polymer, an extract, an exudate, a synthetic polymer, or any combination thereof. Other useful mouth-soluble polymers are known in the art, for example, see Krochta et al., Food Technology (1997) at 51:61-74; Glicksman Food Hydrocolloids CRC 1982; Krochta Edible Coatings and Films to Improve Food Quality Technomic 1994; Industrial Gums Academic 1993; and/or Nussinovitch Water-Soluble Polymer Applications in Foods Blackwell Science 2003, the entire contents of which are hereby incorporated by reference.

In at least one example embodiment, the cellulosic polymer may include, for example, carboxymethyl cellulose (CMC), hydroxypropyl (HPC), hydroxyethyl cellulose (HEC), hydroxypropyl methylcellulose (HPMC), methyl cellulose (MC), or any combination thereof. In at least one example embodiment, the natural polymer may include, for example, a starch, a modified starch, konjac, collagen, inulin, soy protein, whey protein, casein, wheat gluten, or any combination thereof. In at least one example embodiment, the seaweed-derived polymer may include, for example, a carrageenan, an alginate, or a combination of a carrageenan and an alginate. In at least one example embodiment, the carrageenan may include, for example, kappa carrageenan, iota carrageenan, lambda carrageenan, or any combination thereof. In at least one example embodiment, the alginate may include, for example, propylene glycol alginate. In at least one example embodiment, the microbial-derived polymer may include, for example, xanthan, dextran, pullulan, curdlan, gellan, or any combination thereof. In at least one example embodiment, the extract may include, for example, locust bean gum, guar gum, tara gum, gum tragacanth, pectin (e.g., low methoxy and amidated), agar, zein, karaya, gelatin, psyllium seed, chitin, chitosan, or any combination thereof. In at least one example embodiment, the exudate may include, for example, gum acacia (arabic), shellac, or any combination thereof. In at least one example embodiment, the synthetic polymer may include, for example, polyvinyl pyrrolidone, polyethylene oxide, polyvinyl alcohol, or any combination thereof.

As used herein, “mouth-stable” means that the polymer does not appreciably dissolve or disintegrate when exposed to saliva at the normal human body temperature (i.e., 98.6° F.) over a period of about one hour. In at least one example embodiment, the mouth-stable polymer is a biodegradable polymer that is configured to break down over a period of days, weeks, months, or years but does not appreciably break down when held in an oral cavity and exposed to saliva for a period of about one hour. In at least one example embodiment, the mouth-stable polymer is stable within an oral cavity and exposed to saliva at the normal human body temperature for a period of greater than or equal to about 2 hours (e.g., greater than or equal to about 6 hours, greater than or equal to about 12 hours, greater than or equal to about 1 day, or greater than or equal to about 2 days). Accordingly, an pouch filler 104 including a mouth-stable polymer according to at least one example embodiment is configured to remain intact when placed in an adult consumer's mouth. After a period of time, the mouth-stable polymer and any other mouth-stable elements may be removed from the adult consumer's mouth and discarded.

The mouth-stable polymer may be biocompatible and biostable. The mouth-stable polymer may generally be recognized as safe and in compliance with applicable food-contact regulations by an appropriate regulatory agency (e.g., the U.S. Food and Drug Administration (FDA)). In at least one example embodiment, the mouth-stable polymer has a flexural modulus of greater than or equal to about 5 MPa (e.g., greater than or equal to about 10 MPa) when tested according to ASTM Testing Method D790 or ISO 178 at 23° C.

In at least one example embodiment, the mouth-stable polymer may include, for example, a polyurethane, a silicone, a polyester, a polyacrylate, a polyethylene, a polypropylene, a polyetheramide, a polystyrene, a polyvinyl alcohol, a polyvinyl acetate, a polyvinyl chloride, a polybutyl acetate, a butyl rubber, poly(styrene-ethylene-butylene-styrene) (SEBS), poly(styrene-butadiene-styrene) (SBS), poly(styrene-isoprene-styrene) (SIS), any copolymer thereof, or any combination thereof. In at least one example embodiment, the mouth-stable polymer includes a food-grade or medical-grade polymer, such as medical-grade polyurethane.

In at least one example embodiment, the mouth-stable polymer includes, for example, a thermoplastic polymer. The thermoplastic polymer may include a thermoplastic elastomer. In at least one example embodiment, the mouth-stable polymer includes a thermoplastic elastomer meeting the requirements of the FDA-modified ISO 10993, Part 1 “Biological Evaluation of Medical Devices” tests with human tissue contact time of 30 days or less. In at least one example embodiment, the mouth-stable polymer has a shore Hardness of 50D or softer, a melt flow index of about 3 g/10 min at 200° C./10 kg, a tensile strength of greater than or equal to about 10 MPa (using ISO 37), and/or an ultimate elongation of less than about 100% (using ISO 37).

In at least one example embodiment, the pouch filler 104 is free of a sweetener. In at least one example embodiment, the pouch filler 104 includes a sweetener. In at least one example embodiment, the pouch filler 104 includes an encapsulated sweetener. The sweetener may include a synthetic sweetener, a natural sweetener, or a combination of a synthetic sweetener and a natural sweetener.

The natural sweetener may include, for example, a sugar such as monosaccharide, a disaccharide, a polysaccharide, or any combination thereof. The natural sweetener may include, for example, sucrose, honey, a mixture of low-molecular-weight sugars excluding sucrose, glucose (i.e., grape sugar, corn sugar, dextrose), molasses, corn sweetener, glucose syrup (i.e., corn syrup), fructose (i.e., fruit sugar), lactose (i.e., milk sugar), maltose (i.e., malt sugar, maltobiose), sorghum syrup, fruit juice concentrate, or any combination thereof.

In at least one example embodiment, the sweetener includes a sugar alcohol. The sugar alcohol may include, for example, ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof. In at least one example embodiment, the sweetener includes a non-nutritive sweetener. The non-nutritive sweetener may include, for example, stevia, saccharin, aspartame, sucralose, acesulfame potassium, or any combination thereof.

As used herein, the term “nutraceuticals” refers to any ingredient in foods that has a beneficial effect on human health. Nutraceuticals include particular compounds and/or compositions isolated from natural food sources and genetically modified food sources. Suitable nutraceuticals include, without limitation, various phytonutrients derived from natural plants and genetically engineered plants. The nutraceuticals can be included in an amount of about 0.1% to about 5% by weight based on the weight of the composition.

In at least one example embodiment, the pouch filler 104 may include the energizing agent. In at least one example embodiment, the energizing agent includes caffeine, taurine, glucaronalactone, guarana, vitamin B6, vitamin B12, or any combination thereof.

Caffeine, also known as 1,3,7-trimethylxanthine, is a white, odorless, bitter tasting substance. Caffeine occurs naturally in tea, coffee, and chocolate, and is commonly added to soft drinks, energy drinks and some foods. However, because of the bitter taste of caffeine, the flavor of drinks or foods having a relatively high caffeine content can be unappealing. Caffeine may include synthetic caffeine and/or natural caffeine, such as coffee bean-extracted caffeine. In at least one example embodiment, the pouch filler 104 includes caffeine in an amount greater than or equal to about 10 mg (e.g., greater than or equal to about 25 mg, greater than greater than or equal to about 150 mg) The caffeine may be included in an amount less than or equal to about 200 mg (e.g., less than or equal to about 150 mg, less than or equal to about 100 mg, less than or equal to about 75 mg, less than or equal to about 50 mg, or less than or equal to about 25 mg).

The compositions for human consumption may have a relatively high caffeine content so as to provide a consumer with a burst of energy. Moreover, the compositions for human consumption contain about 50 mg to about 200 mg of caffeine or about 75 mg to about 175 mg of caffeine (e.g., 100 mg to about 150 mg of caffeine) so as to provide a burst of energy to the consumer. The composition provides a single serving of a food, drink, oral tobacco product or oral non-tobacco product. A single serving of food can have a weight of about 5 g to about 450 g. A single serving of drink is about 200 mL to about 600 mL. A single serving of an oral pouch product includes one oral pouch product formed as described herein.

Optionally, the composition for human consumption can also include additional energizing ingredients in addition to the caffeine complex. Suitable additional energizing ingredients include, without limitation, taurine, citicoline, and guarana. The additional energizing ingredients can be included in an amount of about 0.1% to about 5% by weight based on the weight of the composition for human consumption.

In at least one example embodiment, the soothing agent includes theanine, melatonin, or both theanine and melatonin. The soothing agent may also include, for example only, chamomile, lavender, jasmine, soursop, cannabidiol, or any combination thereof. The soothing agent can be added as a flavorant and or aroma embedded in the product and/or the package. In at least one example embodiment, the pouch filler 104 includes the soothing agent in an amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the pouch filler 104 includes the soothing agent in an amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

In at least one example embodiment, the pouch filler 104 includes a plasticizer. The plasticizer may include, for example, a monoglyceride, a diglyceride, a triglyceride (e.g., long, medium, and/or short chain), triacetin, propylene glycol, glycerin, vegetable oil, a phthalate, an ester of a polycarboxylic acid with a linear or branched aliphatic alcohol of moderate chain length, or any combination thereof. In at least one example embodiment, the plasticizer may be present in addition to triglycerides and/or other oils in the liquid mixture.

In at least one example embodiment, the pouch filler 104 includes a mouth-soluble fiber. In at least one example embodiment, the mouth-soluble fibers include maltodextrin, psyllium, starch, or any combination thereof. In at least one example embodiment, the mouth-soluble fibers include soluble dietary fibers. In at least one example embodiment pouch filler 104 includes partially-soluble fibers, such as sugar beet fibers.

In at least one example embodiment, the pouch filler 104 includes minerals in addition to any that may be present due to the inclusion, for example, of fruits and/or vegetables. The minerals may include, for example, calcium, magnesium, phosphorus, iron, zinc, iodine, selenium, potassium, copper, manganese, molybdenum, chromium, and any combination thereof. The amount of minerals incorporated into the pouch filler 104 for adult human consumption can be varied according to the type of mineral and the intended adult consumer. The amount of minerals may be formulated to include an amount less than or equal to the recommendations of the United States Department of Agriculture Recommended Daily Allowances.

In at least one example embodiment, the focusing agent includes Ginkgo biloba.

The at least one sensate or chemesthesis agent may include mint, menthol, cinnamon, pepper, jambu, or any combination thereof. The at least one sensate or chemesthesis agent may include any soothing, cooling, and/or warming agent. For example, in some example embodiments, the at least one sensate or chemesthesis agent may include capsaicin, pipeline, alpha-hydroxy-sanshool, and (8)-gingerole, which may be selected so as to provide a warm, tingling or burning sensation. In other example embodiments, the at least one sensate or chemesthesis agent may include menthol, menthyl lactate, WS-3 (N-Ethyl-p menthane-3-carboxamide), WS-23 (2-Isopropyl-N,2,3-trimethylbutyramide) and Evercool 180™ (available from Givaudan SA), which may be selected so as to provide a cooling sensation. The at least one sensate or chemesthesis agent may be included in an amount ranging from about 0.01% by weight to about 5% by weight based on the weight of the pouch filler 104.

The antioxidant may include, for example, vitamin C, vitamin B, magnesium, calcium, or any combination thereof. In at least one example embodiment, the pouch filler 104 may include one or more vitamins in addition to any that may be present due to the inclusion, for example, of fruits and/or vegetables. The one or more additional vitamins may include, for example, vitamin A (retinol), vitamin D (cholecalciferol), vitamin E group, vitamin K group (phylloquinones and menaquinones), thiamine (vitamin B1), riboflavin (vitamin B2), niacin, niacinamide, pyridoxine (vitamin B6 group), folic acid, choline, inositol, vitamin B12 (cobalamins), PABA (para aminobezoic acid), biotin, vitamin C (ascorbic acid), and any combination thereof. The amount of vitamins may be chosen so as to provide an amount less than or equal to the recommendations of the United States Department of Agriculture Recommended Daily Allowances.

Suitable minerals include, without limitation, calcium, magnesium, phosphorus, iron, zinc, iodine, selenium, potassium, copper, manganese, molybdenum, chromium, and mixtures thereof. The amount of minerals incorporated into the composition for human consumption can be varied according to the type of mineral and the intended adult consumer. For example, the amount of minerals may be formulated to include an amount less than or equal to the recommendations of the United States Department of Agriculture Recommended Daily Allowances.

Amino acids can also be included in the composition for human consumption. Suitable amino acids include, without limitation, the eight essential amino acids that cannot be biosynthetically produced in humans, including valine, leucine, isoleucine, lysine, threonine, tryptophan, methionine, and phenylalanine. Examples of suitable amino acids include the non-essential amino acids including alanine, arginine, asparagine, aspartic acid, cysteine, glutamic acid, glutamine, glycine, histidine, proline, serine, and tyrosine. The amino acids can be included in an amount of about 0.1% to about 5% by weight based on the weight of the composition.

In at least one example embodiment, the pouch filler 104 includes a coloring agent. The coloring agent may include a natural colorant, an artificial colorant, or any combination thereof.

In at least one example embodiment, the pouch filler 104 includes the amino acid in an amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the pouch filler 104 includes the amino acid in an amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

In at least one example embodiment, the pouch filler 104 is free of a pH modifier. In at least one example embodiment, the pouch filler 104 includes a pH modifier. The pH modifier may include, for example, ammonium carbonate, ammonium bicarbonate, ammonium hydroxide, calcium carbonate, potassium carbonate, potassium bicarbonate, potassium hydroxide, sodium carbonate, sodium bicarbonate, sodium hydroxide, or any combination thereof.

In at least one example embodiment, the pouch filler 104 includes the pH adjuster in an amount less than or equal to about 2 weight percent (e.g., less than or equal to about 1 weight percent, less than or equal to about 0.5 weight percent, less than or equal to about 0.1 weight percent, or less than or equal to about 0.05 weight percent). In at least one example embodiment, the pouch filler 104 includes the pH adjuster in an amount ranging from about 0.01 weight percent to 2 weight percent.

In at least one example embodiment, the pouch filler 104 is free of a wax. In at least one example embodiment, the pouch filler 104 includes a wax. The wax may include, for example, paraffin, microcrystalline wax, or both paraffin and microcrystalline wax.

In at least one example embodiment, the pouch filler 104 is free of a filler. In at least one example embodiment, the pouch filler 104 includes a filler. The filler may be configured to alter a texture or pliability of the pouch filler 104. The filler may include mouth-soluble elements, mouth-insoluble elements, or both mouth-soluble and mouth-insoluble elements. Mouth-soluble elements may be configured to dissolve or disintegrate when in an adult consumer's mouth so as to render the pouch filler 104 more pliable. The filler may include, for example, dicalcium phosphate, calcium sulfate, a clay, silica, glass particles, glyceryl palmitostearate, sodium stearyl fumarate, talc, or any combination thereof. In at least one example embodiment, certain other compounds or elements or components, including, for example, mouth-soluble fibers, sweeteners, minerals, as described, may be classified as fillers. For example, in at least one example embodiment, cellulosic materials may be present in the pouch filler 104 as fillers in addition to or as an alternative to being carriers for the liquid mixture.

In at least one example embodiment, the pouch filler 104 includes the filler in an amount less than or equal to 20 weight percent (e.g., less than or equal to 15 weight percent, less than or equal to 10 weight percent, less than or equal to 9 weight percent, less than or equal to 8 weight percent, less than or equal to 7 weight percent, less than or equal to 6 weight percent, less than or equal to 5 weight percent, less than or equal to 4 weight percent, less than or equal to 3 weight percent, less than or equal to 2 weight percent, or less than or equal to 1 weight percent). In at least one example embodiment, the pouch filler 104 includes the filler in an amount greater than or equal to about 0 weight percent (e.g., greater than or equal to about 1 weight percent, greater than or equal to about 2 weight percent, greater than or equal to about 3 weight percent, greater than or equal to about 4 weight percent, or greater than or equal to about 5 weight percent). In at least one example embodiment, the pouch filler 104 includes the filler in an amount ranging from about 0 weight percent to about 8 weight percent.

In at least one example embodiment, the pouch filler 104 is free of a flavorant. In at least one example embodiment, the pouch filler 104 includes a flavorant. In at least one example embodiment, the pouch filler 104 includes an encapsulated flavorant. The flavorant may be natural or artificial. The flavorant may include, for example, a fruit flavorant (e.g., bergamot, berry, cherry, lemon, and/or orange), a liquor or liqueur flavorant (e.g., bourbon, cognac, scotch, whiskey, and/or DRAMBUIE brand liqueur), a mint flavorant (e.g., Japanese mint, menthol, peppermint, spearmint, wintergreen, and/or mint oils from a species of the genus Mentha), a floral flavorant (e.g., geranium, lavender, and/or rose), a spice, an herb, or another botanical or botanical-derived flavorant (e.g., anise, apium graveolens, caraway, cardamom, cascarilla, cassia, cinnamon, chamomile, clove, cocoa, coffee, coriander, fennel, ginger, jasmine, licorice, nutmeg, pimenta, sage, sandalwood vanilla, and/or ylang-ylang), honey essence, or any combination thereof. In at least one example embodiment, the flavorant includes bergamot, berry, cherry, lemon, orange, bourbon, cognac, scotch, whiskey, DRAMBUIE brand liqueur, Japanese mint, menthol, peppermint, spearmint, wintergreen, mint oils from a species of the genus Mentha, geranium, lavender, rose, anise, apium graveolens, caraway, cardamom, cascarilla, cassia, cinnamon, chamomile, clove, coffee, coriander, fennel, ginger, jasmine, licorice, nutmeg, pimenta, sage, sandalwood vanilla, ylang-ylang, honey essence, or any combination thereof.

In at least one example embodiment, the pouch filler 104 is free of a carrier. In at least one example embodiment, the pouch filler 104 includes a carrier. The carrier may include a liquid carrier. The liquid carrier may include, for example, water, propylene, glycol, glycerin, ethanol, or any combination thereof.

In at least one example embodiment, the pouch filler 104 is free of an oil. In at least one example embodiment, the oral pouch filler 104 includes an oil. The oil may include, for example, a monoglyceride, a diglyceride, a triglyceride, triacetin, a flavor oil, or any combination thereof. The flavor oil may be or may include a flavorant.

An amount of oil may be selected to achieved a desired texture and/or softness. For example, the softness of the pouch filler 104 may be increased as an amount of the oil in the pouch filler 104 increases. In at least one example embodiment, the pouch filler 104 includes oil in an amount greater than or equal to about 5 weight percent (e.g., greater than or equal to about 8 weight percent, greater than or equal to about 9 weight percent, greater than or equal to about 10 weight percent, greater than or equal to about 11 weight percent, greater than or equal to about 12 weight percent, greater than or equal to about 13 weight percent, or greater than or equal to about 15 weight percent). In at least one example embodiment, the pouch filler 104 includes oil in an amount less than or equal to about 20 weight percent (e.g., less than or equal to about 15 weight percent, less than or equal to about 14 weight percent, less than or equal to about 13 weight percent, less than or equal to about 12 weight percent, less than or equal to about 11 weight percent, less than or equal to about 10 weight percent, or less than or equal to about 8 weight percent). In at least one example embodiment, the pouch filler 104 includes the oil in an amount ranging from about 8 weight percent to about 16 weight percent (e.g., about 10 weight percent to about 14 weight percent, about 11 weight percent to about 13 weight percent, or about 12 weight percent).

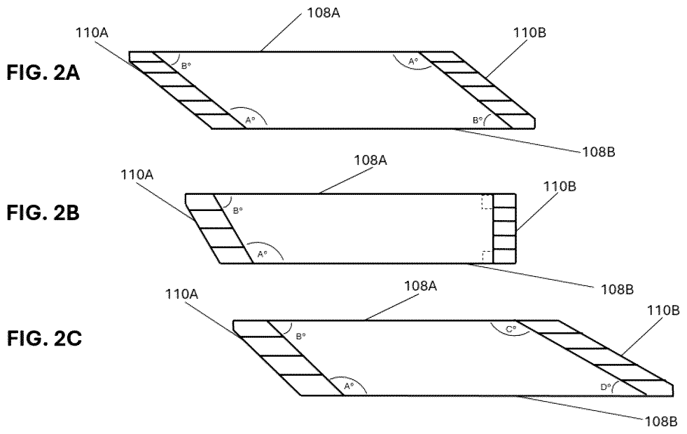

FIG. 2A is a perspective view of an oral pouch product according to at least one example embodiment.

FIG. 2B is a perspective view of an oral pouch product according to at least one example embodiment.

FIG. 2C is a perspective view of an oral pouch product according to at least one example embodiment.

In at least one example embodiment, as shown in FIGS. 2A-2C, the oral pouch product may be substantially the same as those described above with regard to FIGS. 1A-1C, except that the oral pouch product may have blunted or rounded ends or corners to further reduce irritation or discomfort when the oral pouch product is placed in the mouth of an adult consumer. The blunted or rounded ends or corners may be included on one, two, three, or four corners of the pouch.

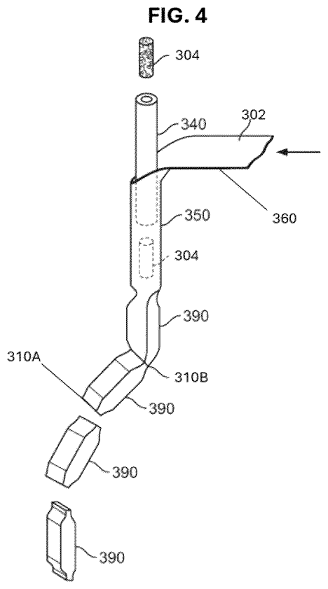

In at least one example embodiment an oral pouch product may be manufactured as depicted in FIG. 4.

A pouch filler 304 can be pouched in a fabric as shown in FIG. 4. As shown, pouch material 302 is formed around tube 340 to form a tube of pouching material 350. The overlapping edge portions of the material 360 can be heat sealed together against tube 340 or between pinch rollers to form the tube 350. A first seal 310A can be made along the tube 350 to form a bottom of a pouch. Pouch filler 304 can be deposited into the partially formed pouch 390 through tube 340. The material 302 can continue to be advanced and a second seal 310B can be made to fully seal the pouch 390 and provide a bottom seal for a subsequent pouch 390. The pouches 390 can be separated along the seal 310B and deposited into a bottom portion 310 of a container. In at least one example embodiment, the sealing and the cutting may be performed simultaneously.

The pinch rollers used to seat the pouch 390 and blades used to separate the pouches along the seal 310B may be angled with respect to the tube 350 such that edges of the pouch 390 along the seal 310A and the seal 310B may angled and form an oblique angle with respect to the path of the tube 350. For example, the edges of the pouch 390 may be angled as described above with regard to FIGS. 1A-2C. Additionally, the pouch filler 304 may be any material or composition as described here and the pouch material 302 may be any material or composition as described herein. For example, as described above with regard to FIGS. 1A-3.

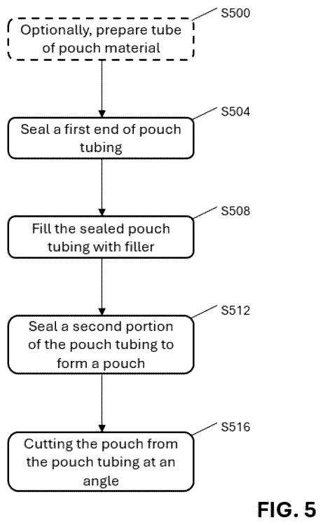

In at least one example embodiment an oral pouch product may be manufactured as shown in FIG. 5.

As shown in FIG. 5, a method of manufacturing an oral pouch product may optionally include preparing a tube of pouching material at S500. The pouch material may be any such material as discussed above with regard to the pouch wrapper 102.

At S504, a method of manufacturing an oral pouch product may include sealing a first end of the pouch tubing. In at least one example embodiment, the sealing may be a heat sealing method as described above with regard to FIG. 4. However, the example embodiments are not limited thereto. For example, the sealing may be performed using an adhesive or any other sealing method known to a person having ordinary skill in the art. In at least one example embodiment, the seal may be at an angle with respect to the tube of pouch material.

At S508, a method of manufacturing an oral pouch product may include filling the sealed pouch tubing with filler. The filler may be any such material as discussed above with regard to the pouch filler 104.

At S512, a method of manufacturing an oral pouch product may include sealing a second portion of the pouch tubing to form a pouch between the first seal and the second seal. In at least one example embodiment, the pouch may have any characteristics as discussed above with regard to the oral pouch product 100. In at least one example embodiment, the second seal may be at an angle with respect to the tube of pouching material. In at least one example embodiment, the second sealing step (S512) of manufacturing a first pouch may also function as the first sealing step (S504) of manufacturing a subsequent pouch. As described above with regard to the first sealing step (504) the second sealing step may be a heat seal, an adhesive seal, or any other sealing method known to a person having ordinary skill in the art.

At S516, a method of manufacturing an oral pouch product may include cutting the pouch from the pouch tubing at an angle with respect to the tube of pouch material. In at least one example embodiment, the pouch may be cut from the tube of pouch material along the second seal. In at least one example embodiment, the pouch may be cut at an angle such that a sealed edge of the pouch may be angled with respect to longitudinal sides of the pouch as described above with regard to FIGS. 1A-2C.

While some example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

Although described with reference to specific examples and drawings, modifications, additions and substitutions of example embodiments may be variously made according to the description by those of ordinary skill in the art. For example, the described techniques may be performed in an order different with that of the methods described, and/or elements such as the described system, architecture, devices, circuit, and the like, may be connected or combined to be different from the above-described methods, or results may be appropriately achieved by other elements or equivalents.

We claim:

1. An oral pouch product for oral use, the oral pouch product comprising:
a filler; and
a pouch enclosing the filler, the pouch including,
a first edge and a second edge extending in parallel,
a first sealed edge extending between the first edge and the second edge, and
a second sealed edge extending between the first edge and the second edge, the second sealed edge spaced apart from the first sealed edge, the first sealed edge oriented such that the first sealed edge forms an acute angle with the first edge and an obtuse angle with the second edge.

2. The oral pouch product of claim 1, wherein the acute angle is 45 degrees or less.

3. The oral pouch product of claim 2, wherein the acute angle ranges from 20 to 45 degrees.

4. The oral pouch product of claim 1, wherein the first sealed edge is parallel to the second sealed edge.

5. The oral pouch product of claim 1, wherein the pouch includes paper.

6. The oral pouch product of claim 1, wherein the filler has a weight of 250 mg to 700 mg.

7. The oral pouch product of claim 1, wherein a distance between the first edge and the second edge is from 8 mm to 12 mm.

8. The oral pouch product of claim 7, where the distance between the first edge and the second edge is less than a length of the first edge.

9. The oral pouch product of claim 1, wherein the pouch has a longest dimension of 29 mm to 36 mm.

10. The oral pouch product of claim 1, wherein the filler includes tobacco.

11. The oral pouch product of claim 1, wherein the filler includes nicotine.

12. The oral pouch product of claim 1, wherein the filler includes caffeine.

13. The oral pouch product of claim 1, wherein the first sealed edge is at an oblique angle with respect to the second sealed edge.

14. The oral pouch product of claim 1, wherein the second sealed edge is perpendicular to the first edge and the second edge.

15. The oral pouch product of claim 1, wherein the second sealed edge is oriented such that the second sealed edge forms an acute angle with the second edge and an obtuse angle with the first edge.

16. An oral pouch product for oral use, the oral pouch product comprising:
a tobacco filler; and
a pouch enclosing the tobacco filler, the pouch including,
a first edge and a second edge extending in parallel,
a first sealed edge extending between the first edge and the second edge, and
a second sealed edge extending between the first edge and the second edge, the second sealed edge spaced apart from the first sealed edge, the first sealed edge oriented such that the first sealed edge forms an acute angle with the first edge and an obtuse angle with the second edge, the first sealed edge extending in parallel to the second sealed edge, and the acute angle ranging from 20 to 45 degrees.

17. The oral pouch product of claim 16, wherein the pouch includes paper.

18. The oral pouch product of claim 16, wherein the tobacco filler has a weight of 250 mg to 700 mg.

19. The oral pouch product of claim 16, wherein a distance between the first edge and the second edge is 8 mm to 12 mm.

20. An oral pouch product for oral use, the oral pouch product comprising:
a filler; and
a pouch enclosing the filler, the pouch including,
a first edge and a second edge extending in parallel,
a first sealed edge extending between the first edge and the second edge, and
a second sealed edge extending between the first edge and the second edge, the second scaled edge spaced apart from the first scaled edge, the first scaled edge oriented such that the first scaled edge forms a non-perpendicular angle with at least one of the first edge or the second edge.
